(19) United States
(12) Patent Application Publication
(10) Pub. No.: US 2008/0033048 A1
(43) Pub. Date: Feb. 7, 2008

(54) PIGGYBACK BIFUNCTIONAL VASODILATORS

(76) Inventors: Daniel J. Smith, Stow, OH (US); Wilmarie Flores-Santana, Silver Spring, MD (US)

Correspondence Address: ROETZEL AND ANDRESS, 222 SOUTH MAIN STREET, AKRON, OH, 44308, US

(21) Appl. No.: 11/750,686

(22) Filed: May 18, 2007

Related U.S. Application Data
(60) Provisional application No. 60/801,736, filed on May 19, 2006.

(51) Int. Cl. A61K 31/13

(52) U.S. Cl. 514/611

(57) ABSTRACT

The present invention generally relates to at least one bifunctional vasodilator composition, and methods for making and using the same. Some embodiments may relate to functionalizing, with nitric oxide, a vasodilator having a secondary amine, thereby forming a diazeniumdiolate derivative of the vasodilator. Such embodiments may be capable of decomposing into nitric oxide and the original vasodilator, or some form thereof having the same or similar vasodilatory properties.

### RELATED APPLICATION DATA

This application claims priority to previously filed U.S. provisional patent application No. 60/801736, filed on May 19, 2006 and entitled “Piggyback Bifunctional Vasodilators”, which is incorporated in its entirety herein by reference.

### BACKGROUND OF THE INVENTION

The present invention generally relates to compositions that break-down into two different vasodilator molecules, including vasodilator compositions that have been modified to include a diazeniumdiolate functionality and compositions that are capable of breaking down into nitric oxide and a vasodilator.

The structures and methods set forth herein are unknown in the art. Thus, the present invention fills a substantial gap in the art.

### SUMMARY OF THE INVENTION

The present invention generally relates to compositions that break-down into two different vasodilator molecules. In some embodiments the present invention relates to compositions comprising vasodilators that have been modified to include a diazeniumdiolate functionality. Furthermore, some embodiments relate to compositions that are capable of breaking down into nitric oxide and at least one vasodilator.

The present invention also relates to a bifunctional vasodilator composition comprising: a first vasodilatory pro-drug moiety; and a second vasodilatory pro-drug moiety, wherein the second moiety is bonded to the first moiety; wherein the vasodilatory composition is capable of decomposing to form a first vasodilatory drug and a second vasodilatory drug. Further, the present invention relates to a process for making a bifunctional vasodilator composition comprising the steps substantially as set forth in the specification and/or attachments thereto.

Still further, the present invention relates to a process for using a vasodilator composition comprising the steps substantially as set forth herein.

### BRIEF DESCRIPTION OF THE FIGURES

FIG. 1 is a graph showing the nitric oxide release profile of a Fendiline diazeniumdiolate as prepared according to Example.

### DETAILED DESCRIPTION OF THE INVENTION

The present invention generally relates to compositions that break-down into two different vasodilator molecules. In some embodiments the present invention relates to compositions comprising vasodilators that have been modified to include a diazeniumdiolate functionality. Furthermore, some embodiments relate to such compositions that are capable of breaking down into nitric oxide and a vasodilator.

Fendiline is a common name for N-(3,3-Diphenylpropyl)-α-methylbenzylamine (CAS No. 13042-18-7). As used herein, the term fendiline includes the free base as well as any salt thereof, including, without limitation, the HCl and/or lactate salt. Similarly, prenylamine is a common name for CAS No. 390-64-7. As used herein, the term prenylamine includes the free base as well as any salt thereof, including, without limitation, the HCl and/or lactate salt.

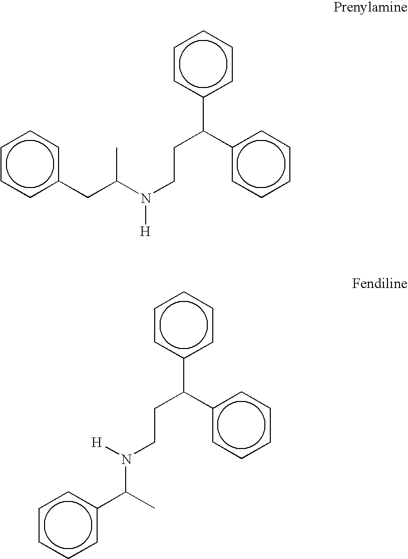

In some embodiments, the present invention relates to derivatizing a first vasodilator with a second vasodilator, e.g. nitric oxide, in a manner in which the derivative can break down into the starting materials and/or some pharmaceutically acceptable form thereof. In embodiments where the second vasodilator is nitric oxide, the first vasodilator includes a secondary amine moiety. Thus, the reversible reaction product comprises a diazeniumdiolate derivative of the first vasodilator.

The first vasodilator can comprise any of a variety of compositions provided it can be reversibly reacted with a second vasodilator. Such compounds include, without limitation, fendiline, prenylamine, and/or any combination thereof. Such compositions also include any pharmacologically similar or analogous derivatives of fendiline, prenylamine, and/or any combination thereof. One of skill in the art would be aware that the vasodilatory effects of fendiline and prenylamine operate differently from that of nitric oxide. The first vasodilator can comprise any appropriate vasodilator regardless of its pharmacological mechanism.

According to some embodiments of the present invention, the pro-drug composition is predominantly lipophilic. Thus, they release nitric oxide slowly because nitric oxide release is activated by contact with water. When the composition eventually comes in contact with water it is capable of releasing nitric oxide quickly. This can occur, for instance, at interfaces between lipids and aqueous media. Alternatively, activation can occur due to small amounts of water dissolved in the lipophilic media containing the composition. In some embodiments, the composition can be transported through the blood stream with lipid transport proteins.

In some embodiments the pro-drug composition can comprise a coating for use on intravascular devices such as balloons, stents, stent grafts, guide wires, and the like. In other embodiments, the pro-drug can comprise treatments for pulmonary hypertension, and/or blood pressure reduction treatments. Still other embodiments can comprise wound care and/or repair treatments, wart removal treatments, skin cancer treatments, antifungal treatments and/or antibacterial treatments. Other embodiments can be used for treating, reducing and/or eliminating vasospasms, particularly such spasms in the brain. In some embodiments the pro-drug can be delivered for the treatment of non-vascular disorders. For instance, the composition can be administered for the purpose using the nitric oxide that is produced for treating viral, bacterial, and/or fungal diseases. In still other embodiments, the present invention is incorporated into a cream, ointment and/or dressing.

Many vasodilators act on smooth muscle through calcium mediated processes. Accordingly, in some embodiments the presence of calcium ion can promote the action of the vasodilator. Thus, in such embodiments it can be beneficial to include calcium in the drug formulation. For instance, in some embodiments the pro-drug can be formulated as a calcium salt.

The pro-drug composition can be delivered in any of a variety of appropriate ways including, without limitation, transdermally, intravascularly, and/or intravenously.

### EXAMPLE 1

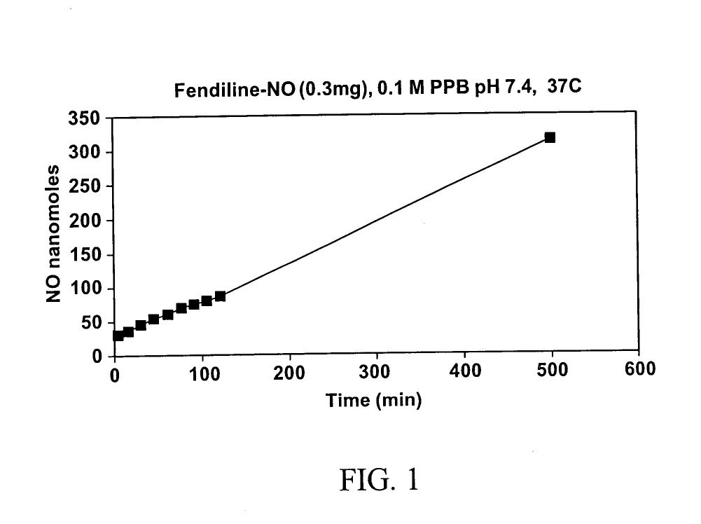

In one embodiment, a diazeniumdiolate derivative of the vasodilator fendiline can be prepared according to the following procedure. Fendiline HCl (CAS No. 13042-18-7) is neutralized with 1M NaOH. The free base of fendiline is extracted in chloroform. After removing the chloroform the sample is modified with nitric oxide in sodium methoxide/methanol. After three days, the modified compound is sampled for UV and nitric oxide analysis (NOA). Then after removal of aqueous sodium methoxide, the product is isolated using chloroform followed by a second extraction in hexane, the resulting powdery product is analyzed for nitric oxide. The nitric oxide release profile of this composition is shown in FIG. 1.

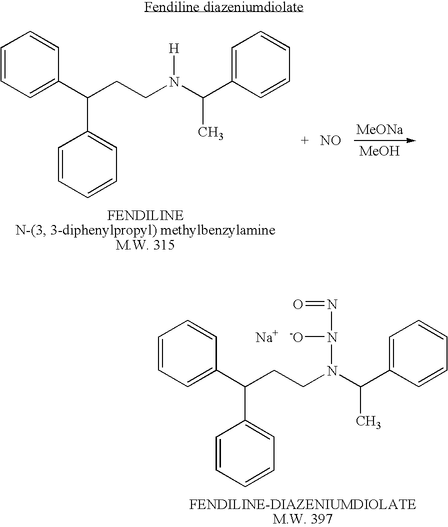

Thus, it can be seen that the objects of the invention have been satisfied by the structure and its method for use presented above. While in accordance with the Patent Statutes, only the best mode and preferred embodiment has been presented and described in detail, it is to be understood that the invention is not limited thereto or thereby. Accordingly, for an appreciation of the true scope and breadth of the invention, reference should be made to the following claims.

What is claimed is:

1. A bifunctional vasodilator composition comprising:
a first vasodilatory pro-drug moiety; and
a second vasodilatory pro-drug moiety, wherein the second moiety is bonded to the first moiety;
wherein the vasodilatory composition is capable of decomposing to form a first vasodilatory drug and a second vasodilatory drug.

2. The composition of claim 1, wherein the first vasodilatory moiety comprises: 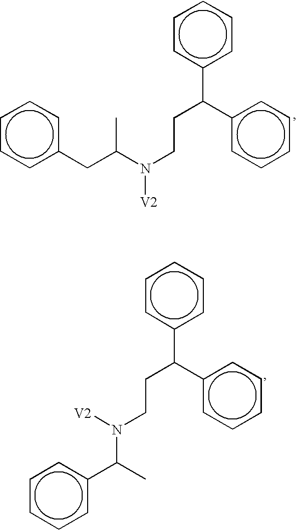
or any combination thereof, wherein V2 comprises the second vasodilatory moiety.

3. The composition of claim 1, wherein the second vasodilatory moiety comprises nitric oxide.

4. The composition of claim 1, wherein the first vasodilatory drug is selected from fendiline, prenylamine, or any combination thereof.

5. The composition of claim 1, wherein the second vasodilatory drug is nitric oxide.

6. A process for making a bifunctional vasodilator composition comprising steps substantially as set forth in the specification and/or attachments thereto.

7. A process for using a vasodilator composition comprising steps substantially as set forth in the specification and/or attachments thereto.
